(19) United States
(12) Patent Application Publication
(10) Pub. No.: US 2010/0063353 A1
(43) Pub. Date: Mar. 11, 2010

(54) ROTATIONAL/LINEAR CONVERTER FOR MEDICAL DEVICE

(75) Inventors: Eliahu Eliachar, Haifa (IL); Nir Lilach, Kfar Yehoshua (IL)

Correspondence Address: Fleit Gibbons Gutman Bongini & Bianco PL, 21355 EAST DIXIE HIGHWAY, SUITE 115, MIAMI, FL, 33180, US

(73) Assignee: ROEI MEDICAL TECHNOLOGIES LTD., Katzin (IL)

(21) Appl. No.: 11/993,868

(22) PCT Filed: Jun. 19, 2006
(86) PCT No.: PCT/IL06/00704

§ 371 Date: Dec. 24, 2007

Related U.S. Application Data
(60) Provisional application No. 60/693,782, filed on Jun. 27, 2005.
(60) Provisional application No. 60/693,783, filed on Jun. 27, 2005.

(51) Int. Cl. A61B 1/00

(52) U.S. Cl. 600/106; 600/118

(57) ABSTRACT

The present invention discloses a surgical equipment, having a proximal portion and a distal portion at least reversibly or temporarily interconnected along a main longitudinal axis (shaft P:D); said proximal portion is insertable into a body cavity, and having at least one manoeuvrable effecter; said effecter is adapted to be either manoeuvred linearly along said axis (linear effecter) or to be manoeuvred rotationally around said axis (rotational effecter); said distal portion comprising a handset located outside the body; said handset is adapted to manoeuvre said effecter with either a linear motion along said axis (linear handset) or a rotational motion around said axis (rotational handset); said effecter comprising a proximal effecting means and a distal converter; said converter translates either linear motion to rotational motion or rotational motion to linear motion; so as said rotational effecter is adaptable to a linear handset, and vice versa, a linear effecter is adaptable to a rotational handset.

### FIELD OF THE INVENTION

The present invention generally relates to a means and methods of translating rotational motion to linear motion and linear motion to rotational motion in surgical equipment. More specifically the invention relates to the transmission of linear motion to the sectioning loop of a resectoscope with a rotational motion handset and the transmission of rotational motion to the sectioning loop of a resectoscope with a linear handset.

### BACKGROUND OF THE INVENTION

Most resectoscopes in use perform the section with linear motion, whereby the forward and backward motion of a high electrical tension loop performs the surgical cut. Other resectoscopes are currently available which perform a similar function by using rotational motion, whereby the twisting motion of the high electrical tension loop performs the cut. In both systems the surgeon controls the motion of the loop from a handset attached to the shaft of the resectoscope.

The handset for the linear motion resectoscope is distinct from the handset of the rotational motion resectoscope. When a surgeon mechanically activates the handset of a linear motion resectoscope, the handset produces a linear motion which is transmitted directly to the resectoscope shaft and hence to the loop. Similarly, when a surgeon mechanically activates the handset of a rotational motion resectoscope the handset produces a rotational motion which is transmitted directly to the resectoscope shaft and hence to the loop. It is not currently possible to produce linear motion of the loop using a rotational motion handset nor is it possible to produce rotational motion of the loop using a linear motion handset. Two separate handsets are required for linear and rotational motion of the loop.

There is a need for transmission mechanisms capable of converting either linear motion produced by the handset into rotational motion of the loop or rotational motion produced by the handset into linear motion of the loop. Thus there remains a long felt need for the present invention relating to a means and method of translating rotational motion to linear motion and linear motion to rotational motion in surgical equipment particularly in resectoscopic applications.

### SUMMARY OF THE INVENTION

It is thus one object of the present invention to disclose a surgical equipment, having a proximal portion and a distal portion at least reversibly or temporarily interconnected along a main longitudinal axis (i.e., shaft P: D). The proximal portion is insertable into a body cavity, and having at least one manoeuvrable effecter. The effecter is adapted to be either manoeuvred linearly along the axis (i.e., linear effecter) or to be manoeuvred rotationally around the axis (i.e., rotational effecter). The distal portion comprising a handset located outside the body. The handset is adapted to manoeuvre said effecter with either a linear motion along said axis (linear handset) or a rotational motion around said axis (i.e., rotational handset). The effecter comprises a proximal effecting means and a distal converter. The converter translates either linear motion to rotational motion or rotational motion to linear motion. In this manner, a rotational effecter is adaptable to a linear handset, and vice versa, a linear effecter is adaptable to a rotational handset.

It is in the scope of the present invention to disclose a linear motion surgical equipment with a rotational motion handset as defined above, comprising a plurality of linear motion connectors and at least one linear motion inhibiting connector such that the effecter performs free linear motion. This equipment may additionally comprise a rotational-linear motion converter for providing transmission of linear motion from a rotational handset, comprising a cylindrical member with at least one helical groove, a shaft which is nested into the cylindrical member, a plurality of pins protruding radially from the surface of the shaft into the helical grooves and an insulating envelope such that rotational motion in the cylindrical member produces linear motion in the nested shaft. The equipment may additionally comprise a rotational-linear motion converter for providing transmission of linear motion from a rotational handset comprising a cylindrical member with at least one helical groove, a shaft which is nested into said cylindrical member, a plurality of pins protruding radially from the surface of the shaft into the helical grooves and an insulating envelope such that rotational motion in the nested shaft linear motion in the cylindrical member.

It is also in the scope of the present invention to disclose a rotational motion surgical equipment with a linear motion handset as defined above, comprising a handset with a protruding shaft, a linear-rotational motion converter, a high tension shaft extending from the linear-rotational motion converter and a working tool connected to said high tension shaft. The activation of the handset produces rotational motion of the, working tool. The high tension shaft and linear-rotational motion converter is possibly attached to the equipment's shaft, additionally comprising a plurality of linear motion connectors and at least one linear motion inhibiting connector such that the high tension shaft performs rotational motion. The equipment may comprise a linear-rotational motion converter for providing transmission of rotational motion from a linear handset comprising a cylindrical member with at least one helical groove, a shaft which is nested into said cylindrical member, a plurality of pins protruding radially from the surface of said shaft into the helical grooves and an insulating envelope such that linear motion in the cylindrical member produces rotational motion in the nested shaft. The equipment may further comprise a linear-rotational motion converter for providing transmission of rotational motion from a linear handset comprising a cylindrical member with at least one helical groove, a shaft which is nested into said cylindrical member, a plurality of pins protruding radially from the surface of the shaft into the helical grooves and an insulating envelope such that linear motion in the nested shaft produces rotational motion in the cylindrical member.

Another object of the present invention is to disclose a method of adapting rotational effecter to a linear handset, and vice versa, adapting a linear effecter to a rotational handset, comprising obtaining a surgical equipment, having a proximal portion and a distal portion at least reversibly or temporarily interconnected along a main longitudinal axis (shaft P:D). The proximal portion is insertable into a body cavity, and having at least one manoeuvrable effecter. The effecter is adapted to be either manoeuvred linearly along said axis (linear effecter) or to be manoeuvred rotationally around said axis (rotational effecter). The distal portion comprising a handset located outside the body. The handset is adapted to manoeuvre said effecter with either a linear motion along the axis (linear handset) or a rotational motion around the axis (rotational handset). The effecter comprises a proximal effecting means and a distal converter. The converter translates either linear motion to rotational motion or rotational motion to linear motion.

It is in the scope of the present invention to disclose a method as defined above, especially adapted for providing transmission of linear motion from a rotational handset by activating the handset thereby rotating the handset shaft and converting the rotational motion in the handset shaft to linear motion in the high tension shaft such that the working tool, attached to the high tension shaft, performs linear motion. The method may additionally include providing free linear motion in the high tension shaft by connecting the high tension shaft to the endoscope shaft by means of a plurality of linear motion connectors which slide freely along the endoscope shaft and connecting the rotational-linear motion converter by means of at least one linear motion inhibiting connector such that the linear motion in transmitted to the high tension shaft. The method may be adapted for converting rotational motion into linear motion by producing rotational motion in cylindrical member with at least one helical groove, introducing a nested shaft into the cylindrical member, providing a plurality of pins protruding radially from the surface of the shaft into the helical grooves such that rotational motion in the cylindrical member exerts a lateral force upon the protruding pins causing them to move with a linear motion. The method may also provided for converting rotational motion into linear motion by producing rotational motion in a shaft, introducing the shaft into a cylindrical member with at least one helical groove, providing a plurality of pins protruding radially from the surface of the shaft into the helical grooves such that rotational motion in the protruding pins exerts a lateral force upon the cylindrical member causing it to move with a linear motion.

It is also in the scope of the present invention to disclose a method as defined in any of the above, especially adapted for providing transmission of rotational motion from a linear handset by activating the handset thereby moving the handset shaft with linear motion and converting the linear motion in the handset shaft to rotational motion in the high tension shaft such that the working tool, attached to the high tension shaft, performs rotational motion. The method may additionally providing free rotational motion in the high tension shaft by connecting the high tension shaft to the endoscope shaft by means of a plurality of linear motion connectors which slide freely along the endoscope shaft and connecting the linear-rotational motion converter to the working tool by means of at least one linear motion inhibiting connector such that the rotational motion in transmitted to the high tension shaft. The method may additionally useful in converting linear motion into rotational motion by producing linear motion in a cylindrical member with at least one helical groove, introducing a nested shaft into said cylindrical member, providing a plurality of pins protruding radially from the surface of the shaft into the helical grooves such that linear motion in the cylindrical member exerts a lateral force upon the protruding pins causing them to move with rotational motion. Lastly, the method may additionally be useful in converting linear motion into rotational motion by producing linear motion in a shaft, introducing said shaft into a cylindrical member with at least one helical groove, providing a plurality of pins protruding radially from the surface of the shaft into the helical grooves such that linear motion in the protruding pins exerts a lateral force upon the cylindrical member causing it to move with rotational motion.

### BRIEF DESCRIPTION OF THE FIGURES

The objects and advantages of various embodiments of the invention will become apparent from the following description when read in conjunction with the accompanying drawings wherein

FIG. 1 schematically represents a full resectoscope apparatus assembled for use according to one embodiment of the current invention;

FIG. 2 schematically represents the end of a resectoscope apparatus with the linear loop assembled for use according to another embodiment of the current invention;

FIG. 3 schematically represents the end of a resectoscope apparatus with the linear loop withdrawn according to another embodiment of the current invention;

FIG. 4 schematically represents a resectoscope apparatus with the outer sheath removed according to another embodiment of the current invention;

FIG. 5 schematically represents the rotational-linear motion converter apparatus attached to a resectoscope according to another embodiment of the current invention;

FIG. 6 schematically represents the rotational-linear motion converter apparatus detached from a resectoscope according to another embodiment of the current invention;

FIG. 7 schematically represents the rotational-linear motion converter apparatus with the insulating sheath removed according to another embodiment of the current invention;

FIG. 8 schematically represents the rotational-linear motion converter apparatus together with the linear movement inhibition ring according to another embodiment of the current invention;

FIG. 9 schematically represents the ends of the rotational shaft and the linear shaft according to another embodiment of the current invention;

FIG. 10 schematically represents the alignment of the rotational shaft and the linear shaft as they are oriented within the rotational-linear motion converter apparatus according to another embodiment of the current invention;

FIG. 11 schematically represents a full resectoscope apparatus assembled for use according to one embodiment of the current invention;

FIG. 12 schematically represents the resectoscope apparatus with the outer sheath removed according to another embodiment of the current invention;

FIG. 13 schematically represents the linear-rotational motion converter apparatus attached to the endoscope shaft according to another embodiment of the current invention;

FIG. 14 schematically represents the linear-rotational motion converter apparatus detached from the endoscope shaft according to another embodiment of the current invention;

FIG. 15 schematically represents the linear-rotational motion converter apparatus with the insulating sheath removed according to another embodiment of the current invention; and,

FIG. 16 schematically represents the alignment of the rotational shaft and the linear shaft as they are oriented within the linear-rotational motion converter apparatus according to another embodiment of the current invention.

### DETAILED DESCRIPTION OF THE EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a means and method of translating rotational motion to linear motion and linear motion to rotational motion in surgical equipment.

The term ‘surgical instrument’ relates hereinafter to any device used in the performance surgical procedure inside a body cavity, outside the body etc. It is in the scope of the present invention wherein the term ‘surgical instrument’ refers to endoscopes in the wide scope of the technology.

Preferably, and in a non-limiting manner, the terms “surgical instrument” or as an example, ‘resectoscope’, relate to one or more of the following either rigid or flexible, disposable or other endoscopes and tools: amnioscope: used to examine the foetus through the cervical canal prior to membrane breakage; angioscope: used to examine the interior of blood vessels; arthroscope: used to examine intraarticular surfaces of joints; bronchoscope: aids in exploring the interior of the bronchi, their branches, and tracheal mucosa (the windpipe tissue lining); choledochoscope: used to examine the bile duct (duct carrying bile from the liver to the gallbladder or from the gallbladder to the small intestine) during an open surgical procedure intraoperatively; colonoscope: used to examine the lower section of the bowel, the large intestine, i.e. the colon; culdoscope: used to examine the pelvis and its structures, which is normally introduced through a small incision in the posterior vaginal cul-de-sac; cystoscope: used to examine the urinary tract and bladder; it employs similar optics to the arthroscope, yet possesses a longer depth of insertion; cystourethroscope: used to examine the urethra, bladder, and distal ureter; encephaloscope: used to examine brain cavities; endoscopic retrograde cholangiopancreatography: used in diagnosis of pancreatic disease through injection of radio-opaque dye into biliary and pancreatic ducts while examining the duodenal area; enteroscope: used to examine the oesophagus, small intestine, and stomach; esophagogastroduodenoscope: used to examine the oesophagus, duodenum, and stomach; esophagoscope: used to examine the channel connecting the pharynx to the stomach; gastroscope: used to examine the stomach lumen; gonioscope: used to examine and help determine the configuration of the angle between the iris and cornea; hysteroscope: used to examine the passage of the uterine cervix and cavity; laparoscope: used to examine the peritoneal cavity through the anterior abdominal wall and is commonly rigid; laryngoscope: used to examine the larynx (the sphincter at the entrance of the trachea); mediastinoscope: used to examine the mediastinum (mass of tissues and organs separating the lungs, i.e. the heart, oesophagus, trachea, etc); often used for visualization of lymph nodes and tumors in the superior mediastinum; nephroscope: used to examine the kidneys, i.e. the renal pelvis, calyces, and upper ureter, it is employed during open procedures intraoperatively; proctoscope: used to examine the rectum; resectoscope: used to perform resections of tissue as a part of a diagnostic or therapeutic procedure; the term ‘resectoscope’ relates hereinafter to any device used in the performance of a biopsy or the removal of tissue from any organs of the body, in particular but not exclusively the resectoscope is used by an urologist to cut tissue from the prostate; rhinoscope: used to examine the nasal cavity; sigmoidoscope: used for direct examination of the sigmoid colon; thoracoscope: used to examine the pleural cavity through an intercostal space (space between adjacent ribs, tilled by intercostals muscles); ureteroscope: used to examine the ureter and/or urethroscope: used to examine the urethra.

The equipment may be further utilized in any non-medical uses for endoscopy, especially in the planning and architectural community have found the endoscope useful for pre-visualization of scale models of proposed buildings and cities (architectural endoscopy), internal inspection of complex technical systems (borescope) etc.

The term ‘rotational motion handset’ relates hereinafter to any device operated by the user mechanically, electrically or by any other means so as to produce rotational motion.

The term ‘linear motion handset’ relates hereinafter to any device operated by the user mechanically, electrically or by any other means so as to produce linear motion.

The term ‘rotational-linear motion converter’ relates hereinafter to any means of converting rotational motion about an axis into linear motion parallel to said axis.

The term ‘linear-rotational motion converter’ relates hereinafter to any means of converting linear motion parallel to an axis into rotational motion about said axis.

The term ‘high tension shaft’ relates hereinafter to any conducting shaft suitable to be held at a high electrical potential.

The term ‘endoscope shaft’ relates hereinafter to any shaft extending from the handset to the tip of the resectoscope, more specifically to a shaft containing an endoscope used to view the working device.

The term ‘plurality’ relates hereinafter to any number greater than or equal to one.

It is according to one embodiment of the current invention to present a linear motion resectoscope with a rotational motion handset comprising the following parts: a handset with a protruding shaft, a rotational-linear motion converter, a high tension shaft extending from the rotational-linear motion converter and a working tool connected to the high tension shaft. The resectoscope is thus assembled such that activation of the handset produces linear motion of the working tool.

It is according to another embodiment of the current invention to present a linear motion resectoscope with a rotational motion handset wherein the high tension shaft and rotational-linear motion converter is attached to an endoscope shaft additionally comprising: a plurality of linear motion connectors and at least one linear motion inhibiting connector such that the high tension shaft performs free linear motion.

It is according to a further embodiment of the current invention to present a rotational-linear motion converter for providing transmission of linear motion from a rotational handset comprising a cylindrical member with at least one helical groove, a shaft which is nested into said cylindrical member, a plurality of pins protruding radially from the surface of said shaft into the helical grooves and an insulating envelope such that rotational motion in the cylindrical member produces linear motion in the nested shaft.

It is according to a farther embodiment of the current invention to present a rotational-linear motion converter for providing transmission of linear motion from a rotational handset comprising a cylindrical member with at least one helical groove, a shaft which is nested into said cylindrical member, a plurality of pins protruding radially from the surface of said shaft into the helical grooves and an insulating envelope such that rotational motion in the nested shaft linear motion in the cylindrical member.

It is according to another embodiment of the current invention to teach a method of providing transmission of linear motion from a rotational handset comprising: activating the handset thereby rotating the handset shaft and converting the rotational motion in the handset shaft to linear motion in the high tension shaft such that the working tool, attached to the high tension shaft, performs linear motion.

It is according to another embodiment of the current invention to teach a method of providing free linear motion in the high tension shaft comprising: connecting the high tension shaft to the endoscope shaft by means of a plurality of linear motion connectors which slide freely along the endoscope shaft and connecting the rotational-linear motion converter by means of at least one linear motion inhibiting connector such that the linear motion in transmitted to the high tension shaft.

It is according to another embodiment of the current invention to teach a method of converting rotational motion into linear motion comprising producing rotational motion in cylindrical member with at least one helical groove, introducing a nested shaft into said cylindrical member, providing a plurality of pins protruding radially from the surface of the shaft into the helical grooves such that rotational motion in the cylindrical member exerts a lateral force upon the protruding pins causing them to move with a linear motion.

It is according to another embodiment of the current invention to teach a method of converting rotational motion into linear motion comprising producing rotational motion in a shaft, introducing said shaft into a cylindrical member with at least one helical groove, providing a plurality of pins protruding radially from the surface of the shaft into the helical grooves such that rotational motion in the protruding pins exerts a lateral force upon the cylindrical member causing it to move with a linear motion.

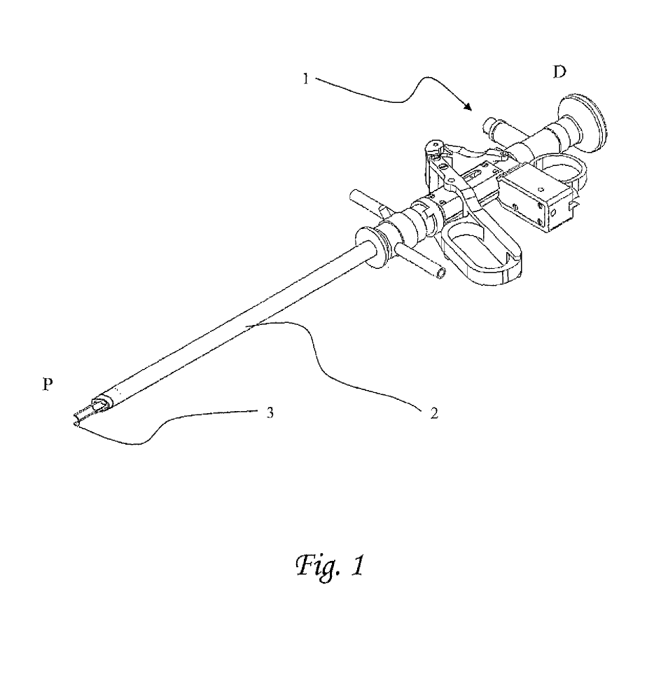

Reference is now made to FIG. 1 schematically representing the full resectoscope apparatus assembled for use according to one embodiment of the current invention. The illustration is equivalently valid for any surgical equipment, having a proximal portion and a distal portion interconnected along a main longitudinal axis P:D, (P for proximal end and D for distal end). The handset, 1, produces rotational motion which is converted into linear motion in the high electrical tension loop, 3. In this diagram the rotational-linear motion converter apparatus is hidden beneath the outer sheath, 2.

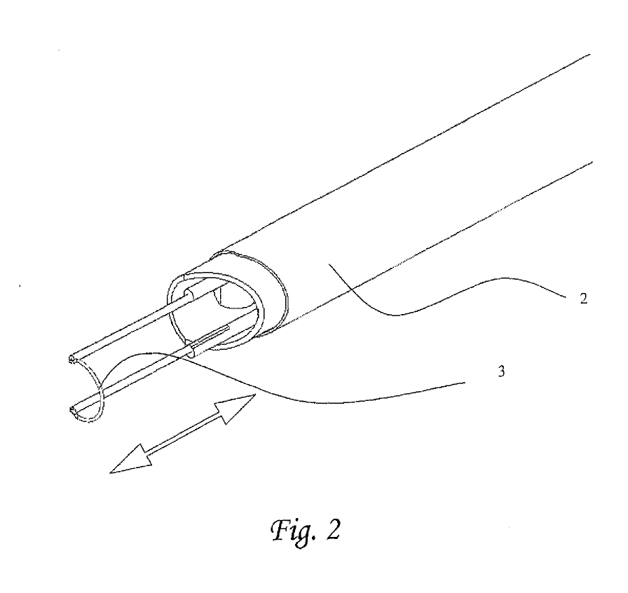

Reference is now made to FIG. 2 which schematically represents the end of the resectoscope apparatus with the linear loop, 3, assembled for use according to another embodiment of the current invention. The direction of the motion of the high electrical tension loop is shown by the arrow.

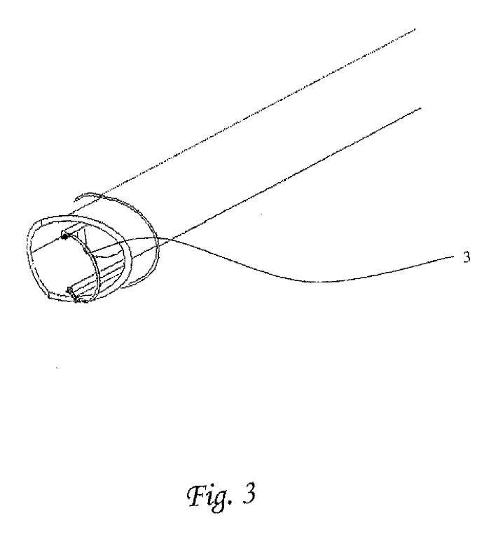

Reference is now made to FIG. 3 which schematically represents the end of the resectoscope apparatus with the linear loop, 3, withdrawn into the outer sheath according to another embodiment of the current invention. This represents the extremity of linear motion which can be produced by the loop in this direction.

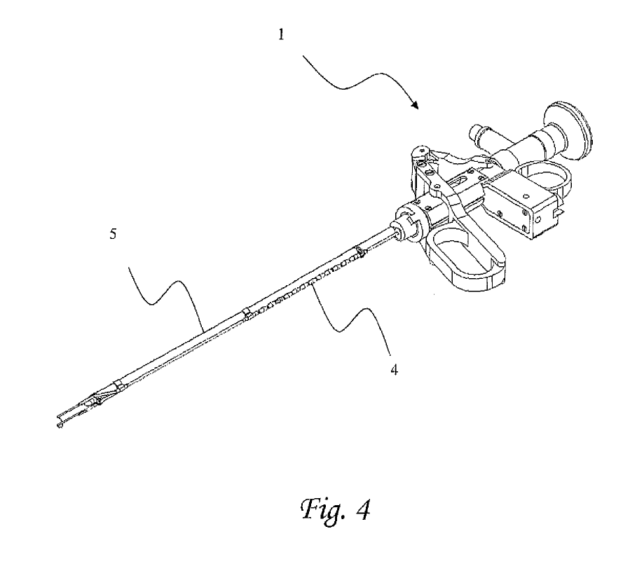

Reference is now made to FIG. 4 which schematically represents the resectoscope apparatus with the outer sheath removed according to another embodiment of the current invention. Here the transmission mechanism is visible. The rotational-linear motion converter apparatus, 4, is attached to the endoscope shaft, 5.

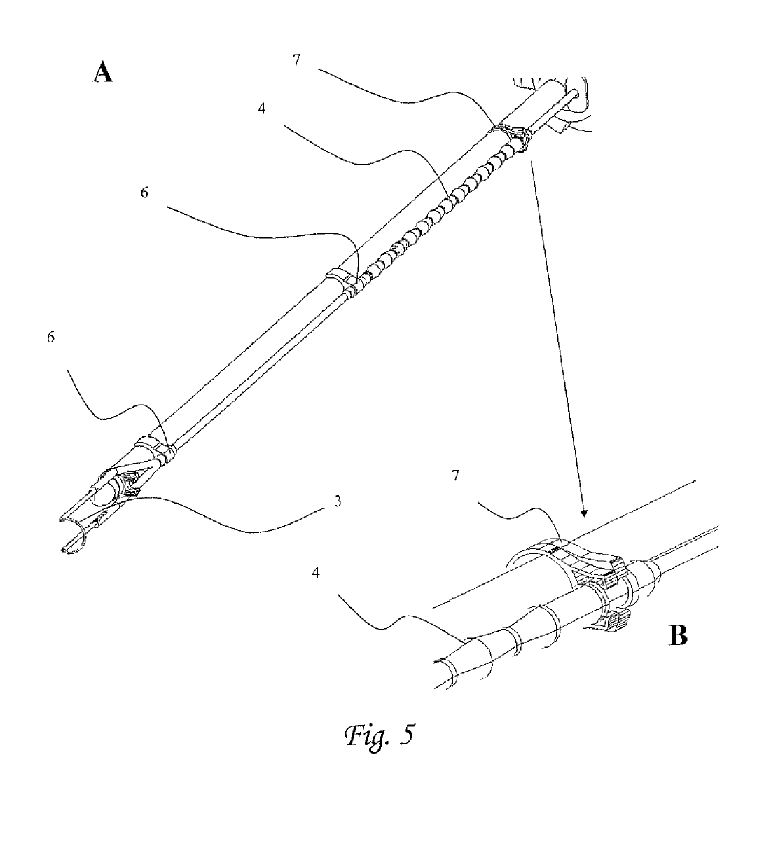

Reference is now made to FIG. 5 which schematically represents the rotational-linear motion converter apparatus attached to the endoscope according to another embodiment of the current invention. In diagram A it can be seen that the rotational-linear motion converter is attached to the endoscope shaft at one end by a linear motion connector, 6, which allows the high electrical tension shaft to move with linear motion relative to the endoscope shaft, a second linear motion connector, secures the high electrical tension shaft closer to the loop. The other end of the rotational-linear motion converter is attached to the endoscope shaft by a linear movement inhibiting connector, 7, which prevents the rotational handset shaft from moving in a linear direction relative to the rotational-linear motion converter. This can be seen in more detail in diagram B. The rotational-linear motion converter is covered in this diagram by an insulating sheath, 4.

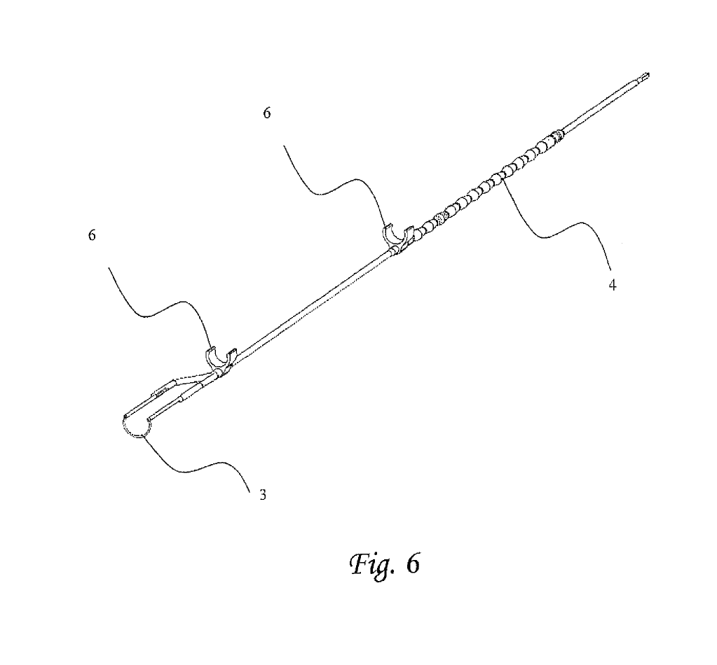

Reference is now made to FIG. 6 which schematically represents the rotational-linear motion converter apparatus detached from the endoscope shaft according to another embodiment of the current invention. Here the semi-circular structure of the linear motion connectors is visible. The rotational-linear motion converter is covered in this diagram by an insulating sheath, 4.

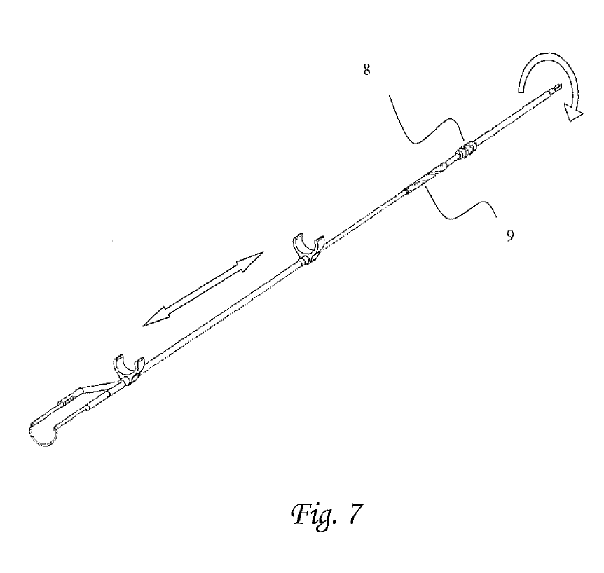

Reference is now made to FIG. 7 which schematically represents the rotational-linear motion converter apparatus with the insulating sheath removed according to another embodiment of the current invention. The motion of each section is signified by the arrows. Here the rotational-linear motion converter, 9, is visible as is the linear motion inhibition ring, 8.

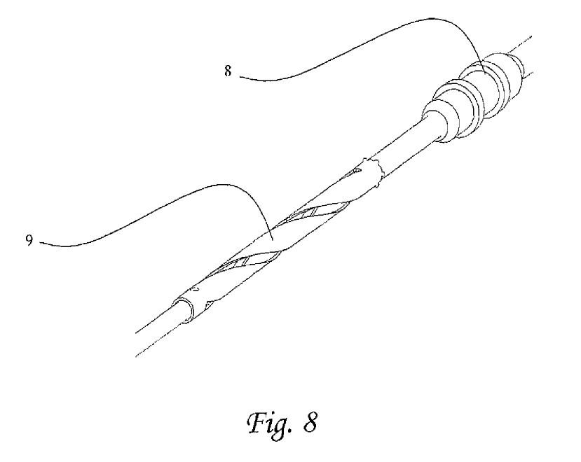

Reference is now made to FIG. 8 which schematically represents the rotational-linear motion converter apparatus, 9, together with the linear motion inhibition ring, 8, according to another embodiment of the current invention. The helical groove provides a track for a linear motion pin situated on the end of the high tension shaft such that when the rotational-linear motion converter rotates the linear motion pin pushes the high tension shaft in a linear direction.

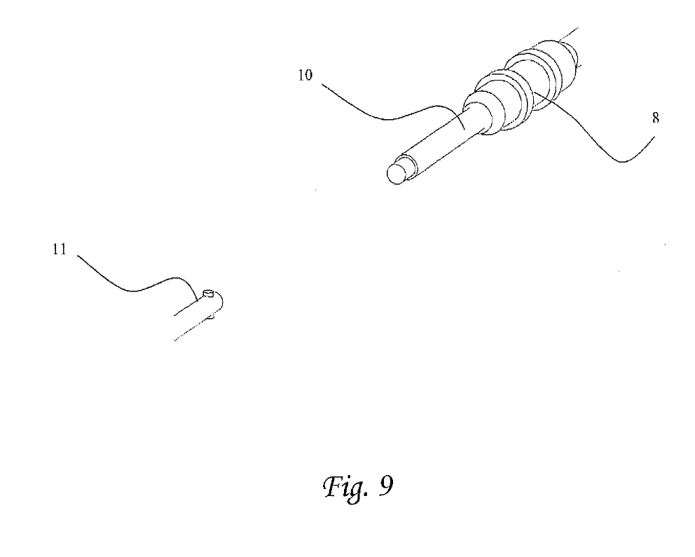

Reference is now made to FIG. 9 which schematically represents the ends of the rotational handset shaft, 10, and the high tension shaft, 11, according to another embodiment of the current invention.

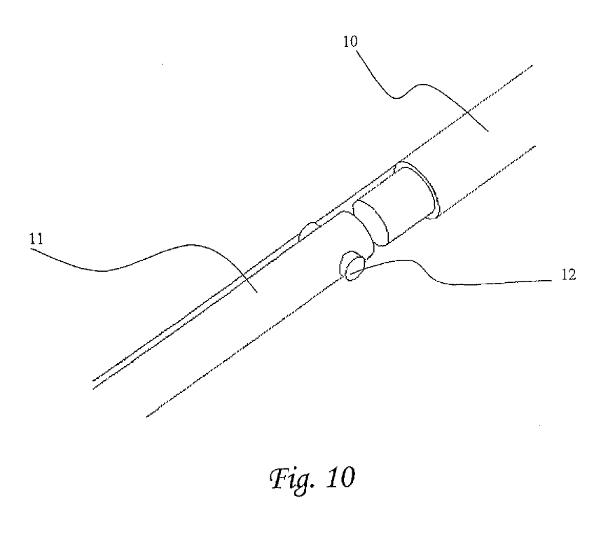

Reference is now made to FIG. 10 which schematically represents the alignment of the rotational handset shaft, 10, and the high tension shaft, 11, as they are oriented within the rotational-linear motion converter apparatus according to another embodiment of the current invention. The linear motion pin, 12, would be situated in the helical groove which is connected to the rotating shaft, 10 such that the pin, 12, and so the high tension shaft, 11, is pushed along in a linear motion.

It is according to one embodiment of the current invention to present a rotational motion resectoscope with a linear motion handset comprising the following parts: a handset with a protruding shaft, a linear-rotational motion converter, a high tension shaft extending from the linear-rotational motion converter and a working tool connected to said high tension shaft. The resectoscope is thus assembled such that activation of the handset produces rotational motion of the working tool.

It is according to another embodiment of the current invention to present a rotational motion resectoscope with a linear motion handset wherein the high tension shaft and linear-rotational motion converter is attached to an endoscope shaft additionally comprising: a plurality of linear motion connectors and at least one linear motion inhibiting connector such that the high tension shaft performs rotational motion.

It is according to a further embodiment of the current invention to present a linear-rotational motion converter for providing transmission of rotational motion from a linear handset comprising a cylindrical member with at least one helical groove, a shaft which is nested into said cylindrical member, a plurality of pins protruding radially from the surface of said shaft into the helical grooves and an insulating envelope such that linear motion in the cylindrical member produces rotational motion in the nested shaft

It is according to a further embodiment of the current invention to present a linear-rotational motion converter for providing transmission of rotational motion from a linear handset comprising a cylindrical member with at least one helical groove, a shaft which is nested into said cylindrical member, a plurality of pins protruding radially from the surface of said shaft into the helical grooves and an insulating envelope such that linear motion in the nested shaft produces rotational motion in the cylindrical member.

It is according to another embodiment of the current invention to teach a method of providing transmission of rotational motion from a linear handset comprising: activating the handset thereby moving the handset shaft with linear motion and converting the linear motion in the handset shaft to rotational motion in the high tension shaft such that the working tool, attached to the high tension shaft, performs rotational motion.

It is according to another embodiment of the current invention to teach a method of providing free rotational motion in the high tension shaft comprising: connecting the high tension shaft to the endoscope shaft by means of a plurality of linear motion connectors which slide freely along the endoscope shaft and connecting the linear-rotational motion converter to the working tool by means of at least one linear motion inhibiting connector such that the rotational motion in transmitted to the high tension shaft.

It is according to another embodiment of the current invention to teach a method of converting linear motion into rotational motion comprising producing linear motion in a cylindrical member with at least one helical groove, introducing a nested shaft into said cylindrical member, providing a plurality of pins protruding radially from the surface of the shaft into the helical grooves such that linear motion in the cylindrical member exerts a lateral force upon the protruding pins causing them to move with rotational motion.

It is according to another embodiment of the current invention to teach a method of converting linear motion into rotational motion comprising producing linear motion in a shaft, introducing said shaft into a cylindrical member with at least one helical groove, providing a plurality of pins protruding radially from the surface of the shaft into the helical grooves such that linear motion in the protruding pins exerts a lateral force upon the cylindrical member causing it to move with rotational motion.

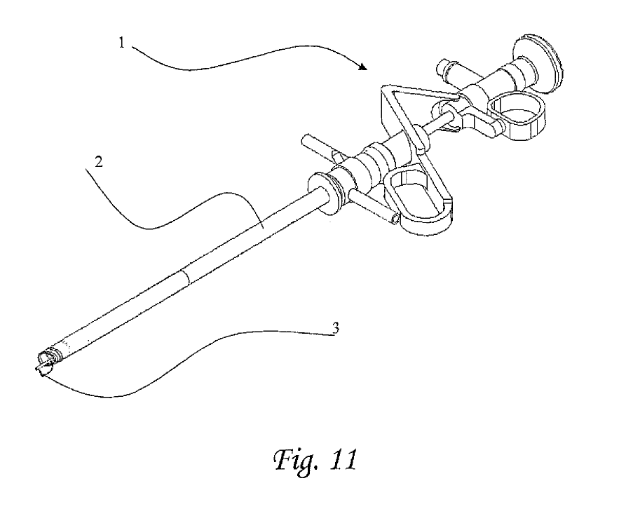

Reference is now made to FIG. 11 schematically representing the full resectoscope apparatus assembled for use according to one embodiment of the current invention. The handset, 1, produces linear motion which is converted into rotational motion in the high electrical tension loop, 3. In this diagram the linear-rotational motion converter apparatus is hidden beneath the outer sheath, 2.

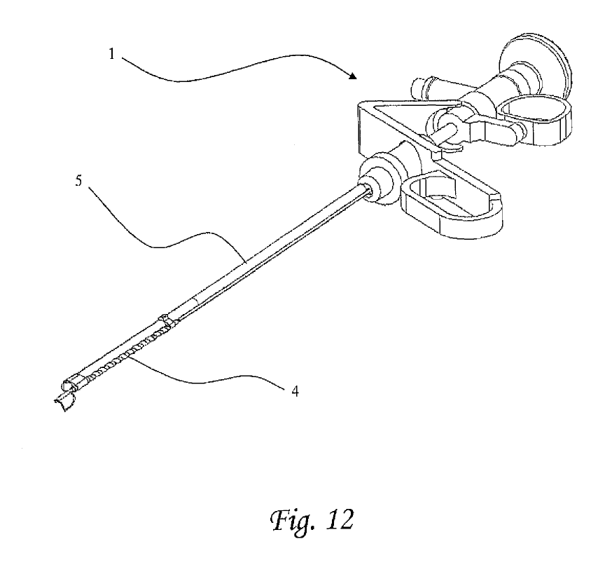

Reference is now made to FIG. 12 which schematically represents the resectoscope apparatus with the outer sheath removed according to another embodiment of the current invention. Here the transmission mechanism is visible. The linear-rotational motion converter apparatus, 4, is attached to the endoscope shaft, 5.

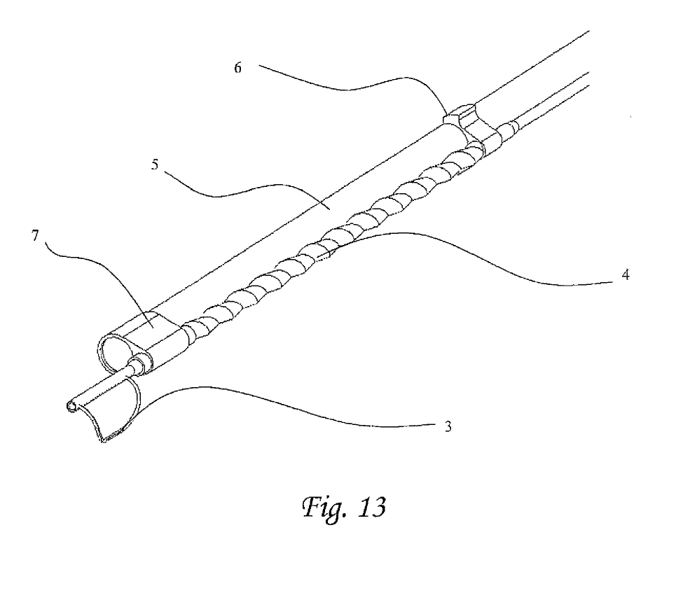

Reference is now made to FIG. 13 which schematically represents the resectoscope apparatus with the outer sheath removed according to another embodiment of the current invention. Here the transmission mechanism is visible. The linear-rotational motion converter apparatus, 4, is attached to the endoscope shaft, 5, at one end by a linear motion connector, 6, which allows the high electrical tension shaft to move with linear motion relative to the endoscope shaft. The other end of the linear-rotational motion converter is attached to the endoscope shaft by a linear movement inhibiting connector, 7, which prevents the rotational handset shaft from moving in a linear direction relative to the linear-rotational motion converter.

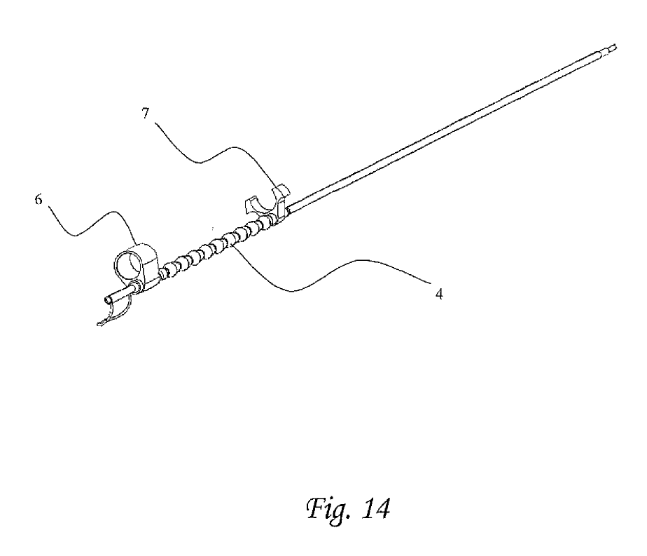

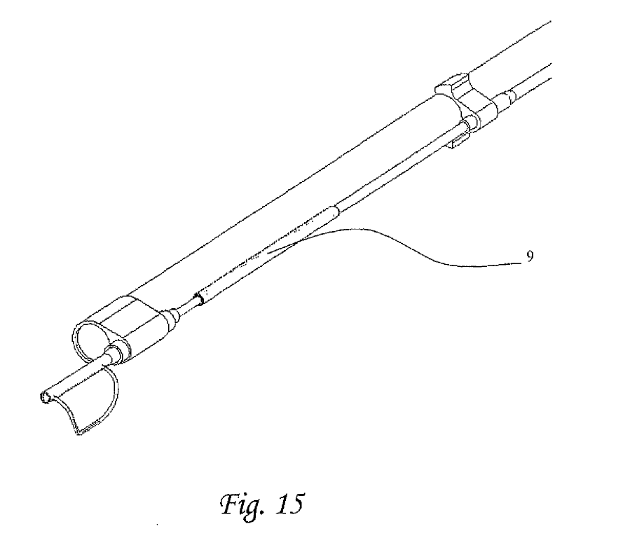

Reference is now made to FIG. 14 which schematically represents the linear-rotational motion converter apparatus detached from the endoscope shaft according to another embodiment of the current invention. Here the semi-circular structure of the linear motion connector, 7, is visible. The linear-rotational motion converter is covered in this diagram by an insulating sheath, 4. Reference is now made to FIG. 15 which schematically represents the linear-rotational motion converter apparatus with the insulating sheath removed according to another embodiment of the current invention. Here the linear-rotational motion converter, 9, is visible.

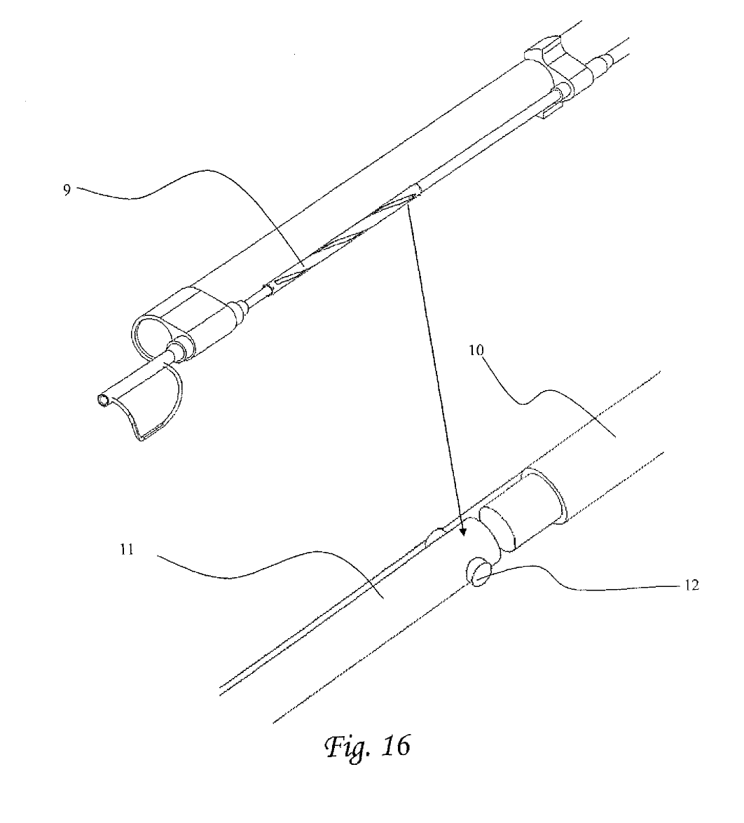

Reference is now made to FIG. 16 which schematically represents the linear-rotational motion converter apparatus, 9. The helical groove provides a track for a rotational motion pin situated at the end of the rotational shaft, 11, leading to the high tension loop. When the linear-rotational motion converter moves with linear motion, the rotational motion pin turns the high tension shaft with rotational motion.

1. A surgical equipment, having a proximal portion and a distal portion at least reversibly or temporarily interconnected along a main longitudinal axis (shaft P:D); said proximal portion is insertable into a body cavity, and having at least one manoeuvrable effecter; said effecter is adapted to be either manoeuvred linearly along said axis (linear effecter) or to be manoeuvred rotationally around said axis (rotational effecter); said distal portion comprising a handset located outside the body; said handset is adapted to manoeuvre said effecter with either a linear motion along said axis (linear handset) or a rotational motion around said axis (rotational handset); said effecter comprising a proximal effecting means and a distal converter; said converter translates either linear motion to rotational motion or rotational motion to linear motion; so as said rotational effecter is adaptable to a linear handset, and vice versa, a linear effecter is adaptable to a rotational handset.

2. A linear motion surgical equipment with a rotational motion handset according to claim 1, comprising a plurality of linear motion connectors and at least one linear motion inhibiting connector such that the effecter performs free linear motion.

3. The linear motion surgical equipment with a rotational motion handset according to claim 2, additionally comprising a rotational-linear motion converter for providing transmission of linear motion from a rotational handset comprising a cylindrical member with at least one helical groove, a shaft which is nested into said cylindrical member, a plurality of pins protruding radially from the surface of said shaft into the helical grooves and an insulating envelope such that rotational motion in the cylindrical member produces linear motion in the nested shaft.

4. The linear motion surgical equipment with a rotational motion handset according to claim 2, additionally comprising a rotational-linear motion converter for providing transmission of linear motion from a rotational handset comprising a cylindrical member with at least one helical groove, a shaft which is nested into said cylindrical member, a plurality of pins protruding radially from the surface of said shall into the helical grooves and an insulating envelope such that rotational motion in the nested shaft linear motion in the cylindrical member.

5. A rotational motion surgical equipment with a linear motion handset according to claim 1, comprising a handset with a protruding shaft, a linear-rotational motion converter, a high tension shaft extending from the linear-rotational motion converter and a working tool connected to said high tension shaft; such that activation of the handset produces rotational motion of the working tool.

6. The rotational motion surgical equipment with a linear motion handset according to claim 5, wherein. the high tension shaft and linear-rotational motion converter is attached to said equipment's shaft, additionally comprising a plurality of linear motion connectors and at least one linear motion inhibiting connector such that the high tension shaft performs rotational motion.

7. The rotational motion surgical equipment with a linear motion handset according to claim 5, additionally comprising a linear-rotational motion converter for providing transmission of rotational motion from a linear handset comprising a cylindrical member with at least one helical groove, a shaft which is nested into said cylindrical member, a plurality of pins protruding radially from. the surface of said shaft into the helical grooves and an insulating envelope such that linear motion in the cylindrical member produces rotational motion in the nested shall.

8. The rotational motion surgical equipment with a linear motion handset according to claim 5, additionally comprising a linear-rotational motion converter for providing transmission of rotational motion from a linear handset comprising a cylindrical member with at least one helical groove, a shaft which is nested into said cylindrical member, a plurality of pins protruding radially from the surface of said shaft into the helical grooves and an insulating envelope such that linear motion in the nested shaft produces rotational motion in the cylindrical member.

9. The surgical equipment as defined in claim 1, wherein the surgical equipment is an endoscope, especially resectoscope.

10. A method of adapting rotational effecter to a linear handset, and vice versa, adapting a linear effecter to a rotational handset, comprising obtaining a surgical equipment, having a proximal portion and a distal portion at least reversibly or temporarily interconnected along a main longitudinal axis (shaft P:D); said proximal portion is insertable into a body cavity, and having at least one manoeuvrable effecter; said effecter is adapted to be either manoeuvred linearly along said axis (linear effecter) or to be manoeuvred rotationally around. said axis (rotational effecter); said distal portion comprising a handset located outside the body; said handset is adapted to manoeuvre said effecter with either a linear motion along said axis (linear handset) or a rotational motion around said axis (rotational handset); said effecter comprising a proximal effecting means and a distal converter; said converter translates either linear motion to rotational motion or rotational motion to linear motion.

11. A method according to claim 10 for providing transmission of linear motion from a rotational handset by activating the handset thereby rotating the handset shaft and converting the rotational motion in the handset shaft to linear motion in the high tension shaft such that the working tool, attached to the high tension shaft, performs linear motion.

12. The method according to claim 11, additionally providing free linear motion in the high tension shaft by connecting the high tension shaft to the endoscope shaft by means of a plurality of linear motion connectors which slide freely along the endoscope shaft and connecting the rotational-linear motion converter by means of at least one linear motion inhibiting connector such that the linear motion in transmitted to the high tension shaft.

13. The method according to claim 11 for converting rotational motion into linear motion by producing rotational motion in cylindrical member with at least one helical groove, introducing a nested shaft into said cylindrical member, providing a plurality of pins protruding radially from the surface of the shaft into the helical grooves such that rotational motion in the cylindrical member exerts a lateral force upon the protruding pins causing them to move with a linear motion.

14. The method, according to claim 11, of converting rotational motion into linear motion by producing rotational motion in a shaft, introducing said shaft into a cylindrical member with at least one helical groove, providing a plurality of pins protruding radially from the surface of the shaft into the helical grooves such that rotational motion in the protruding pins exerts a lateral force upon the cylindrical member causing it to move with a linear motion.

15. The method according to claim 11 for providing transmission of rotational motion from a linear handset by activating the handset thereby moving the handset shaft with linear motion and converting the linear motion in the handset shaft to rotational motion in the high tension shaft such that the working tool, attached to the high tension shaft, performs rotational motion.

16. The method according to claim 15, additionally providing free rotational motion in the high tension shaft by connecting the high tension shaft to the endoscope shaft by means of a plurality of linear motion connectors which slide freely along the endoscope shaft and connecting the linear-rotational motion converter to the working tool by means of at least one linear motion inhibiting connector such that the rotational motion in transmitted to the high tension shaft.

17. The method according to claim 15, additionally converting linear motion into rotational motion by producing linear motion in a cylindrical member with at least one helical groove, introducing a nested shaft into said cylindrical member, providing a plurality of pins protruding radially from the surface of the shaft into the helical grooves such that linear motion in the cylindrical member exerts a lateral force upon the protruding pins causing them to move with rotational motion.

18. The method according to claim 15, additionally converting linear motion into rotational motion by producing linear motion in a shaft, introducing said shaft into a cylindrical member with at least one helical groove, providing a plurality of pins protruding radially from the surface of the shaft into the helical grooves such that linear motion in the protruding pins exerts a lateral force upon the cylindrical member causing it to move with rotational motion.
